(19) United States
(12) Patent Application Publication
(10) Pub. No.: US 2003/0064039 A1
(43) Pub. Date: Apr. 3, 2003

(54) Foundation composition comprising interference pigments

(76) Inventors: Richard Kolodziej, Paris (FR); Jean-Christophe Simon, Paris (FR)

Correspondence Address: Thomas L. Irving, FINNEGAN, HENDERSON, FARABOW,, GARRETT & DUNNER, L.L.P., 1300 I Street, N.W., Washington, DC, 20005-3315, US

(21) Appl. No.: 10/233,293

(22) Filed: Sep. 3, 2002

Related U.S. Application Data
(60) Provisional application No. 60/326,431, filed on Oct. 3, 2001.

(30) Foreign Application Priority Data
Sep. 3, 2001 (FR) 01 11374

(51) Int. Cl. A61K 7/021

(52) U.S. Cl. 424/63

(57) ABSTRACT

The invention relates to a composition comprising, in a physiologically acceptable medium, goniochromatic pigments exhibiting a colour effect and an additional colouring agent, the colour effect of the composition having a natural look.

The invention also relates to a skin makeup process.

The present invention relates to a cosmetic composition containing goniochromatic pigments, and more especially to a makeup composition for the skin of the human face or body.

The composition according to the invention is a coloured makeup composition, for instance a product for the complexion (foundation), a face powder, an eye-shadow, a concealer product, a blusher or a body makeup product. The composition is generally used in unmodified form for application to the skin, but it may also be applied over a makeup that has already been applied to the skin, for example to modify the makeup (the composition is applied as a surface product commonly known as a top coat).

The makeup composition may also be applied to makeup accessories (supports), for instance pastilles or patches that adhere to the skin or the lips (such as beauty spots).

It is known practice to use interference pigments in makeup products, especially for their particular colour effects (WO 96/03962 for a particular interference pigment, or DE 199 07 313). Makeup compositions containing interference pigments may also contain particular dyestuffs, for instance lakes (see JP-A-2000-34203 or JP-A-2000-34204).

The aim of the present invention is to propose a makeup composition presenting a makeup whose colour corresponds to the colour of the skin, thus giving a natural colour effect. Another aim of the invention is to provide a makeup composition which, after application to the skin, can modify the visual appearance of the contours of the face, thus modifying the appearance of the volumes of the face that are visible to the naked eye; this particular makeup effect corresponds to a facial sculpturing effect, also known as “morphing”.

The inventors have discovered that such a composition may be obtained by combining particular interference pigments, namely goniochromatic pigments, with an additional colouring agent in specific amounts. The composition produces a makeup that has a facial sculpturing effect, while at the same time maintaining a natural look.

The composition applied to the skin achieves good camouflaging of skin imperfections.

More specifically, the invention relates to a composition comprising, in a cosmetically acceptable medium, goniochromatic pigments exhibiting a colour effect and an additional dyestuff, characterized in that the goniochromatic pigments and the additional colouring agent are present in the composition in a weight ratio of goniochromatic pigments to active material of the additional colouring agent ranging from 0.1 to 2.

A subject of the invention is also a cosmetic skin makeup process comprising the application to the skin of a composition as defined above.

A subject of the invention is also a made-up support, such as the makeup accessories mentioned above, comprising a makeup that may be obtained according to the makeup process defined above and applied to the said support.

In the present patent application, the expression “physiologically acceptable medium” means a medium that is compatible with human keratin materials such as the skin, the nails, the hair, the eyelashes or the eyebrows, as a cosmetic medium.

A goniochromatic pigment is a pigment that can produce different colours depending on the incidence of the light and the angle of observation.

More specifically, such a pigment makes it possible to obtain, when the cosmetic composition is spread onto a support, a colour path in the a*b* plane of the 1976 CIE (International Commission on Illumination) calorimetric space which corresponds to a variation Dh of the hue angle h of at least 20° when the angle of observation is varied relative to the normal between 0° and 80°, for an angle of light incidence of 45°.

The colour path may be measured, for example, using an Instrument Systems spectrogonioreflectometer of reference GON 360 Goniometer, after the cosmetic composition has been spread in fluid form to a thickness of 300 μm using an automatic spreader onto an Erichsen contrast card of reference Typ 24/5, the measurement being performed on the black background of the card.

For the purposes of the present invention, a goniochromatic pigment makes it possible to observe a colour change, also known as a “colour flop”, as a function of the angle of observation, greater than the change that may be encountered with nacres.

Preferably, the goniochromatic pigments are goniochromatic pigments of multilayer interference structure.

The multilayer structure of the goniochromatic pigments may include at least two layers, each layer, which may or may not be independent of the other layer(s), being made of at least one material chosen from the group consisting of the following materials: MgF₂, CeF₃, ZnS, ZnSe, Si, SiO₂, Ge, Te, Fe₂O₃, Pt, Va, Al₂O₃, MgO, Y₂O₃, S₂O₃, SiO, HfO₂, ZrO₂, CeO₂, Nb₂O₅, Ta₂O₅, TiO₂, Ag, Al, Au, Cr, Cu, Rb, Ti, Ta, W, Zn, MoS₂, cryolite, alloys and polymers, and combinations thereof.

Preferably, the goniochromatic pigment of multilayer interference structure according to the invention is chosen from the group consisting of the following commercial goniochromatic pigments: Infinite Colors manufactured or sold by the company Shiseido, Sicopearl Fantastico manufactured or sold by the company BASF, Colorstream manufactured or sold by the company Merck, Colorglitter manufactured or sold by the company 3M, Chromaflair manufactured or sold by the company Flex, Xirallic and Xirona manufactured or sold by the company Merck, and mixtures thereof.

Accordingly, the multilayer structure may be essentially mineral or organic. Depending on the thickness of each of the various layers, different colours are obtained.

The goniochromatic pigments of multilayer interference structure according to the invention are especially those described in the following documents: U.S. Pat. No. 3,438,796, EP-A-227 423, U.S. Pat. No. 5,135,812, EP-A-170 439, EP-A-341 002, U.S. Pat. No. 4,930,866, U.S. Pat. No. 5,641,719, EP-A-472 371, EP-A-395 410, EP-A-753 545, EP-A-768 343, EP-A-571 836, EP-A-708 154, EP-A-579 091, U.S. Pat. No. 5,411,586, U.S. Pat. No. 5,364,467, WO-A-97/39066, DE-A-4 225 031, WO 95/17479 (BASF), DE-A-196 14 637, and combinations thereof. They are in the form of flakes, of metallized colour.

For example, the multilayer interference structure is chosen from the group consisting of the following structures: Al/SiO₂/Al/SiO₂/Al; Cr/MgF₂/Al/MgF₂/Cr; MOS₂/SiO₂/Al/SiO₂/MoS₂; Fe₂O₃/SiO₂/Al/SiO₂/Fe₂O₃; Fe₂O₃/SiO₂/Fe₂O₃/SiO₂/Fe₂O₃; MoS₂/SiO₂/mica oxide/SiO₂/MoS₂; Fe₂O₃/SiO₂/mica oxide/SiO₂/Fe₂O₃, and combinations thereof.

In general, the structure is composed of alternate layers of low optical index and of high optical index.

The goniochromatic pigments may be present in the composition according to the invention in a content ranging from 0.01% to 50% by weight, preferably from 0.1% to 30% by weight and better still from 0.3% to 20% by weight relative to the total weight of the composition.

The colouring agent present in the composition according to the invention is different from the goniochromatic pigments described above. The additional colouring agent may be chosen from pigments, nacres and dyes, and mixtures thereof.

The colouring agent may comprise at least one pigment. The term “pigments” should be understood as meaning white or coloured, mineral or organic particles of any shape, which are insoluble in the physiological medium, and which are intended to colour the composition. The terms “nacres” and “nacreous pigments” should be understood as meaning iridescent particles of any shape, produced especially by certain molluscs in their shell, or alternatively synthesized.

The pigments may be present in the composition in a proportion of from 0 to 30% (especially 0.01% to 30%), preferably from 0.01% to 15% by weight and better still from 0.02% to 5% by weight relative to the weight of the composition.

The pigments may be white or coloured, and mineral and/or organic. Among the mineral pigments that may be mentioned are titanium dioxide, optionally surface-treated, zirconium oxide and cerium oxide, and also zinc oxide, iron oxide (black, yellow or red), chromium oxide, manganese violet, ultramarine blue, chromium hydrate and ferric blue, and metal powders, for instance aluminium powder or copper powder, and mixtures thereof.

Among the organic pigments that may be mentioned are carbon black, pigments of D & C type, and lakes based on cochineal carmine or on barium, strontium, calcium or aluminium, and mixtures thereof.

The colouring agent may also comprise at least one nacre or nacreous pigment.

The nacres may be present in the composition in a proportion of from 0 to 25% (especially 0.01% to 25%) by weight, preferably from 0.01% to 15% by weight and better still from 0.02% to 5% by weight relative to the total weight of the composition.

The nacreous pigments may be chosen from white nacreous pigments such as mica coated with titanium or with bismuth oxychloride, coloured nacreous pigments such as titanium mica coated with iron oxides, titanium mica coated especially with ferric blue or with chromium oxide, titanium mica coated with an organic pigment of the abovementioned type, and also nacreous pigments based on bismuth oxychloride, and mixtures thereof.

The composition of the invention may, inter alia, comprise dyes that are soluble in physiological medium, and in particular liposoluble or water-soluble dyes.

The colouring agent may also comprise a colorant chosen from water-soluble or liposoluble dyes or alternatively colouring polymers. The colorant may be present in the composition in a content of colouring active material ranging from 0 to 6% (especially 0.01% to 6%) by weight and preferably ranging from 0.01% to 3% by weight relative to the total weight of the composition.

The liposoluble dyes are, for example, soybean oil, Sudan brown, DC Yellow 11, DC Orange 5, quinoline yellow, Sudan Red III (CTFA name D&C red 17), lutein, quinizarine green (CTFA name DC green 6), Alizurol SS purple (CTFA name DC violet No. 2), carotenoid derivatives, for instance lycopene, beta-carotene, bixin or capsanthin, and/or mixtures thereof.

Among the water-soluble dyes that may be mentioned are dyeing plant extracts such as, for example, Aleurites moluccana Willd, Alkanna tinctoria Tausch, Areca catechu L., Arrabidaea chica E. and B., Bixa orellana L (annatto), Butea monosperma Lam, Caesalpina echinata Lam, Caesalpina sappan L., Calophyllum inophyllum L., Carthamus tinctorius L., Cassia alata L., Chrozophora tinctoria L., Crocus sativus L., Curcuma longa L., Diospyros gilletii Wild, Eclipta prostrata L., Gardenia erubescens Stapf. and Hutch., Gardenia terniflora Schum. and Thonn., Genipa americana L., Genipa brasiliensis L., Guibourtia demeusei (Harms) J. Leon, Haematoxylon campechianum L., Helianthus annuus, Humiria balsamifera (Aubl.) St-Hil., Isatis tinctoria L., Mercurialis perenis, Monascus purpureus, Monascus ruber, Monascus pilosus, Morus nigra L., Picramnia spruceana, Pterocarpus erinaceus Poir., Pterocarpus soyauxii Taub., Rocella tinctoria L., Rothmannia whitfieldii (Lindl.) Dand., Schlegelia violacea (Aubl.) Griseb., Simira tinctoria Aublet, Stereospermum kunthianum Cham., Symphonia globulifera L., Terminalia catappa L., sorghum, Aronia melanocarpa, naphthoquinones including lawsone, derived from Lawsonia inermis L., also known as henna, or from Impatiens balsamina, red wood extracts as described in document WO 98/44902, beetroot juice, the disodium salt of suschin, anthocyans, for instance extracts of red berries, dihydroxyacetone, monocarbonyl or polycarbonyl derivatives such as isatin, alloxan, ninhydrin, glyceraldehyde, mesotartaric aldehyde, pyrazoline-4,5-dione derivatives, and mixtures thereof, these skin-colouring agents optionally being combined with direct dyes or indole derivatives, and/or mixtures thereof.

These dyeing plant extracts may be in the form of a lyophilizate, a paste or a solution: generally, the leaves of the dyeing plant are ground to obtain a powder. This powder is dissolved in an aqueous phase for several hours. The mixture is subsequently centrifuged and then filtered. The filtrate obtained is frozen and then lyophilized.

The colouring agent may also comprise a colouring polymer, ie a polymer comprising at least one organic colouring group. The colouring polymer generally contains less than 10% by weight of colorant relative to the total weight of the polymer.

The colouring polymer may be of any chemical nature, especially a polyester, polyamide, polyurethane, polyacrylic, poly(meth)acrylic, polycarbonate, polymers of natural origin, for instance cellulose polymers or chitosan polymers, or mixtures thereof, and preferably polyester or polyurethane polymers.

The colouring polymer may comprise a colouring group [lacuna] may be grafted, especially by covalent bonding, onto the polymer chain, as described in documents WO-A-96/29046, WO-A-92/01022, WO-A-90/07558 and BE-A-609 054.

In particular, the colouring polymer may be a copolymer based on at least two different monomers, at least one of which is an organic colouring monomer.

The monomers of the colouring polymer may be chosen from anthraquinones, methines, bis-methines, aza-methines, arylidenes, 3H-dibenzo[7,i-j]isoquinolines, 2,5-diarylaminoterephthalic acids and esters thereof, phthaloylphenothiazines, phthaloylphenoxazines, phthaloylacridone, anthrapyrimidines, anthrapyrazoles, phthalocyanins, quinophthalones, indophenols, perinones, nitroarylamines, benzodifurans, 2H-l-benzopyran-2-ones, quinophthalones, perylenes, quinacridones, triphenodioxazines, fluoridines, 4-amino-1,8-naphthalimides, thioxanthrones, benzanthrones, indanthrones, indigos, thioindigos, xanthenes, acridines, azines and oxazines.

Colouring monomers are described especially in documents U.S. Pat. No. 4,267,306; U.S. Pat. No. 4,359,570; U.S. Pat. No. 4,403,092, U.S. Pat. No. 4,617,373; U.S. Pat. No. 4,080,355; U.S. Pat. No. 4,740,581; U.S. Pat. No. 4,116,923; U.S. Pat. No. 4,745,173; U.S. Pat. No. 4,804,719; U.S. Pat. No. 5,194,463; U.S. Pat. No. 5,804,719; WO-A-92/07913.

Polymeric colorants are described especially in documents U.S. Pat. No. 4,804,719; U.S. Pat. No. 5,032,670; U.S. Pat. No. 4,999,418; U.S. Pat. No. 5,106,942; U.S. Pat. No. 5,030,708; U.S. Pat. No. 5,102,980; U.S. Pat. No. 5,043,376; U.S. Pat. No. 5,194,463; WO-A-92/07913; WO-A-97/24102, the content of which is incorporated into the present patent application by reference.

Sulphopolyester colouring-polymers such as those described in document WO-A-97/24102 are preferably used.

The colouring polymers may be present in the composition according to the invention in a content ranging from 0% to 50% by weight (0.01% to 50%), preferably ranging from 0.5% to 25% by weight and better still ranging from 0.2% to 20% by weight relative to the total weight of the composition.

According to the invention, the goniochromatic pigments and the additional colouring agent are present in the composition according to the invention in a weight ratio of goniochromatic pigments to active material of the additional colouring agent of less than or equal to 2, preferably ranging from 0.1 to 1.5 and even more preferably ranging from 0.5 to 1.5, for example equal to about 1 (ie ranging from 0.9 to 1.1).

In addition to the additional colouring agent, the composition according to the invention may also contain fillers. The term “fillers” should be understood as meaning colourless or white, mineral or synthetic particles of any form, which are insoluble in the medium of the composition irrespective of the temperature at which the composition is manufactured. These fillers serve especially to modify the rheology or texture of the composition.

The fillers may be mineral or organic of any form, platelet-shaped, spherical or oblong-shaped, irrespective of the crystallographic form (for example leaflet, cubic, hexagonal, orthorhombic, etc.). Mention may be made of talc, mica, silica, kaolin, polyamide powder (Nylon®)(Orgasol® from Atochem), poly-β-alanine powder and polyethylene powder, tetrafluoroethylene polymer (Teflon®) powders, lauroyllysine, starch, boron nitride, polymeric hollow microspheres such as those of polyvinylidene chloride/acrylonitrile, for instance Expancel® (Nobel Industrie), acrylic acid polymers (Polytrap from the company Dow Corning), and silicone resin microbeads (for example Tospearls® from Toshiba), elastomeric polyorganosiloxane particles, precipitated calcium carbonate, magnesium carbonate and magnesium hydrocarbonate, hydroxyapatite, hollow silica microspheres (Silica Beads® from Maprecos), glass microcapsules and ceramic microcapsules; metal soaps derived from organic carboxylic acids containing from 8 to 22 carbon atoms and preferably from 12 to 18 carbon atoms, such as, for example, zinc, magnesium or lithium stearate, zinc laurate or magnesium myristate. The fillers may be present in a proportion of from 0 to 90% by weight, preferably 0.01% to 50% by weight and better still from 0.02% to 30% by weight relative to the total weight of the composition, especially of the base and/or surface composition.

The composition of the invention may comprise a particulate phase comprising the pigments and/or nacres and/or fillers as defined above, which may be present in a proportion of 0 to 98% (especially 0.01% to 98%), preferably from 0.01% to 30% and better still from 0.02% to 20% relative to the total weight of the composition.

The composition according to the invention may comprise a hydrophilic cosmetic medium or a lipophilic medium.

The composition may comprise water or a mixture of water and hydrophilic organic solvents, for instance alcohols and especially linear or branched lower monoalcohols containing from 2 to 5 carbon atoms, for instance ethanol, isopropanol or n-propanol, polyols, for instance glycerol, diglycerol, propylene glycol, sorbitol, pentylene glycol and polyethylene glycols. The hydrophilic phase may also contain C₂ ethers and C₂-C₄ aldehydes that are hydrophilic. The water or the mixture of water and hydrophilic organic solvents may be present in the composition according to the invention, or one of the base and/or surface compositions, in a content ranging from 0% to 90% (especially 0.1% to 90%) by weight and preferably from 0% to 60% by weight (especially 0.1% to 60% by weight) relative to the total weight of the composition.

The composition may also comprise a fatty phase consisting especially of fatty substances that are liquid at room temperature (in general 25° C.) and/or fatty substances that are solid at room temperature, such as waxes, pasty fatty substances and gums, and mixtures thereof. This fatty phase may also contain lipophilic organic solvents.

As fatty substances that are liquid at room temperature, often known as oils, which may be used in the invention, mention may be made of: hydrocarbon-based oils of animal origin, such as perhydrosqualene; hydrocarbon-based plant oils such as liquid triglycerides of fatty acids containing from 4 to 10 carbon atoms, for instance heptanoic or octanoic acid triglycerides, or alternatively sunflower oil, corn oil, soybean oil, grape seed oil, sesame seed oil, apricot oil, macadamia oil, castor oil, avocado oil, caprylic/capric acid triglycerides, jojoba oil or karite butter; linear or branched hydrocarbons of mineral or synthetic origin, such as liquid paraffins and derivatives thereof, petroleum jelly, polydecenes or hydrogenated polyisobutene such as parleam; synthetic esters and ethers, especially of fatty acids such as, for example, purcellin oil, isopropyl myristate, 2-ethylhexyl palmitate, 2-octyldodecyl stearate, 2-octyldodecyl erucate or isostearyl isostearate; hydroxylated esters, for instance isostearyl lactate, octyl hydroxystearate, octyldodecyl hydroxystearate, diisostearyl malate, triisocetyl citrate and fatty alkyl heptanoates, octanoates and decanoates; polyol esters, for instance propylene glycol dioctanoate, neopentyl glycol diheptanoate or diethylene glycol diisononanoate; pentaerythritol esters; fatty alcohols containing from 12 to 26 carbon atoms, for instance octyldodecanol, 2-butyloctanol, 2-hexyldecanol, 2-undecylpentadecanol or oleyl alcohol; partially hydrocarbon-based and/or silicone-based fluoro oils; silicone oils, for instance volatile or non-volatile, linear or cyclic polymethylsiloxanes (PDMS), which may be liquid or pasty at room temperature, for instance cyclomethicones, dimethicones, optionally comprising a phenyl group, for instance phenyltrimethicones, phenyltrimethylsiloxydiphenylsiloxanes, diphenylmethyldimethyltrisiloxanes, diphenyldimethicones, phenyldimethicones or polymethylphenylsiloxanes; mixtures thereof.

These oils may be present in a content ranging from 0.01% to 90% and better still from 0.1% to 85% by weight relative to the total weight of the composition.

The composition according to the invention may also comprise one or more cosmetically acceptable organic solvents (acceptable tolerability, toxicology and feel). These solvents may be present in a content ranging from 0 to 90% and better still from 0 to 60% by weight relative to the total weight of the composition, better still from 0.1% to 30%.

As solvents that may be used in the composition of the invention, mention may be made of acetic acid esters, for instance methyl, ethyl, butyl, amyl, 2-methoxyethyl or isopropyl acetate; ketones, for instance methyl ethyl ketone or methyl isobutyl ketone; hydrocarbons, for instance toluene, xylene, hexane or heptane; aldehydes containing from 5 to 10 carbon atoms; ethers containing at least 3 carbon atoms; and mixtures thereof.

The composition of the invention may also advantageously comprise a fatty substance that is solid or pasty at room temperature, for instance gums or waxes. The waxes may be hydrocarbon-based waxes, fluoro waxes and/or silicone waxes and may be of plant, mineral, animal and/or synthetic origin. In particular, the waxes have a melting point of greater than 25° C. and better still greater than 45° C.

As waxes that may be used in the composition of the invention, mention may be made of beeswax, carnauba wax, candelilla wax, paraffin, microcrystalline waxes, ceresin or ozokerite; synthetic waxes, for instance polyethylene waxes or Fischer-Tropsch waxes, and silicone waxes, for instance alkyl- or alkoxydimethicones containing from 16 to 45 carbon atoms.

The gums are generally polydimethylsiloxanes (PDMS) of high molecular weight or cellulose gums or polysaccharides, and the pasty substances are generally hydrocarbon-based compounds, for instance lanolins and derivatives thereof, or alternatively PDMSs.

The nature and amount of the solid substances depend on the desired mechanical properties and textures. As a guide, the composition may contain from 0.01% to 50% by weight and better still from 1% to 30% by weight of waxes relative to the total weight of the composition.

The composition according to the invention may especially be in the form of a suspension, a dispersion, a solution, a gel, an emulsion, especially an oil-in-water (O/W) or water-in-oil (W/O) emulsion, or a multiple emulsion (W/O/W or polyol/O/W or O/W/O), in the form of a cream, a paste, a mousse, a dispersion of vesicles, especially of ionic or nonionic lipids, a two-phase or multi-phase lotion, a spray, a powder, a paste, especially a soft paste (especially a paste with a dynamic viscosity at 25° C. of about 0.1 to 40 Pa.s at a shear rate of 200 s⁻¹, after 10 minutes of measurement in cone/plate geometry). The composition may have an organic continuous phase, which may especially be anhydrous.

A person skilled in the art may select the appropriate presentation form, and also the method for preparing it, on the basis of his general knowledge, taking into account firstly the nature of the constituents used, especially their solubility in the support, and secondly the intended use of the composition.

The composition according to the invention may also contain ingredients commonly used in cosmetics, such as vitamins, thickeners (especially clays, which are optionally modified), trace elements, softeners, sequestering agents, fragrances, acidifying or basifying agents, preserving agents and UV screening agents, and mixtures thereof.

Needless to say, a person skilled in the art will take care to select this or these optional additional compounds, and/or the amount thereof, such that the advantageous properties of the composition according to the invention are not, or are not substantially, adversely affected by the envisaged addition.

The composition of the invention may be obtained according to the preparation processes conventionally used in cosmetics or dermatology.

The examples of compositions below are given for illustrative purposes and with no limiting nature.

### EXAMPLE 1

| | |
|---|---|
| octyl palmitate | 52.4% |
| microcrystalline wax | 12.1% |
| dimethicone 300 cSt | 0.3% |
| acrylate copolymers (Expancel ®) | 1.5% |
| Nylon powder 1-2 | 13.0% |
| goniochromatic pigments sold under the | 10.0% |
| name “Sicopearl” by the company BASF | |
| yellow iron oxide | 0.47% |
| red iron oxide | 0.40% |
| black iron oxide | 0.18% |
| titanium dioxide | 9.45% |

The foundation applied to the face gives a makeup of natural appearance with facial sculpturing effects.

### EXAMPLE 2

A foundation was prepared, comprising:

| | |
|---|---|
| octyl palmitate | 52.4% |
| microcrystalline wax | 12.1% |
| dimethicone 300 cSt | 0.3% |
| acrylate copolymers (Expancel ®) | 1.5% |
| Nylon powder 1-2 | 13.0% |
| goniochromatic pigments sold under the | 12.0% |
| name “Sicopearl” by the company BASF | |
| yellow iron oxide | 0.38% |
| red iron oxide | 0.15% |
| black iron oxide | 0.32% |
| titanium dioxide | 7.65% |

The foundation applied to the face gives a makeup of natural appearance with facial sculpturing effects.

1. Composition comprising, in a physiologically acceptable medium, goniochromatic pigments and an additional colouring agent, characterized in that the goniochromatic pigments and the additional colouring agent are present in the composition in a weight ratio of goniochromatic pigments to active material of the additional colouring agent ranging from 0.1 to 2.

2. Composition according to claim 1, such that the goniochromatic pigments are pigments of multilayer interference structure comprising at least two layers, each layer being made of at least one material chosen from the group consisting of the following materials: MgF₂, CeF₃, ZnS, ZnSe, Si, SiO₂, Ge, Te, Fe₂O₃, Pt, Va, Al₂O₃, MgO, Y₂O₃, S₂O₃, SiO, HfO₂, ZrO₂, CeO₂, Nb₂O₅, Ta₂O₅, TiO₂, Ag, Al, Au, Cr, Cu, Rb, Ti, Ta, W, Zn, MoS₂, cryolite, alloys and polymers, and combinations thereof.

3. Composition according to either of the preceding claims, such that the goniochromatic pigments are pigments of multilayer interference structure chosen from the group consisting of the structures: Al/SiO₂/Al/SiO₂/Al; Cr/MgF₂/Al/MgF₂/Cr; MoS₂/SiO₂/Al/SiO₂/MoS₂; Fe₂O₃/SiO₂/Al/SiO₂/Fe₂O₃; Fe₂O₃/SiO₂/Fe₂O₃/SiO₂/Fe₂O₃; MoS₂/SiO₂/mica oxide/SiO₂/MoS₂; Fe₂O₃/SiO₂/mica oxide/SiO₂/Fe₂O₃, and combinations thereof.

4. Composition according to one of the preceding claims, such that the goniochromatic pigments are present in a content ranging from 0.01% to 50% by weight relative to the total weight of the composition.

5. Composition according to one of the preceding claims, such that the goniochromatic pigments are present in a content ranging from 0.1% to 30% by weight relative to the total weight of the composition.

6. Composition according to one of the preceding claims, such that the goniochromatic pigments are present in a content ranging from 0.3% to 20% by weight relative to the total weight of the composition.

7. Composition according to one of the preceding claims, such that the additional colouring agent is chosen from the group formed by pigments, nacres, water-soluble or liposoluble dyes, and colouring polymers, and mixtures thereof.

8. Composition according to one of the preceding claims, such that the additional colouring agent comprises at least one pigment chosen from titanium dioxide, zirconium oxides, cerium oxides, zinc oxides, iron oxides, chromium oxides, manganese violet, ultramarine blue, chromium hydrate, ferric blue, aluminium powder, copper powder, carbon black, pigments of D & C type, and lakes based on cochineal carmine or on barium, strontium, calcium or aluminium, and mixtures thereof.

9. Composition according to claim 7 or 8, such that the pigments are present in a content ranging from 0.01% to 30% by weight relative to the weight of the composition.

10. Composition according to one of claims 7 to 9, such that the pigments are present in a content ranging from 0.01% to 15% by weight relative to the weight of the composition.

11. Composition according to one of claims 7 to 10, such that the pigments are present in a content ranging from 0.02% to 5% by weight relative to the weight of the composition.

12. Composition according to one of the preceding claims, such that the colouring agent comprises at least one nacre chosen from mica coated with titanium or with bismuth oxychloride, titanium mica coated with iron oxides, titanium mica coated with ferric blue or with chromium oxide, titanium mica coated with an organic pigment, and nacreous pigments based on bismuth oxychloride, and mixtures thereof.

13. Composition according to claim 12, such that the nacres are present in a content ranging from 0.01% to 25% by weight relative to the weight of the composition.

14. Composition according to claim 12 or 13, such that the nacres are present in a content ranging from 0.01% to 15% by weight relative to the weight of the composition.

15. Composition according to one of claims 12 to 14, such that the nacres are present in a content ranging from 0.02% to 5% by weight, relative to the weight of the composition.

16. Composition according to one of the preceding claims, such that the colouring agent comprises a dyestuff chosen from water-soluble or liposoluble dyes and colouring polymers, and is present in a content of colouring active material ranging from 0.01% to 6% by weight relative to the total weight of the composition.

17. Composition according to any one of the preceding claims, such that the goniochromatic pigments and the additional colouring agent are present in a weight ratio of interference particles to active material of the additional colouring agent ranging from 0.1 to 1.5.

18. Composition according to any one of the preceding claims, such that the goniochromatic pigments and the additional colouring agent are present in a weight ratio of goniochromatic pigments to active material of the additional colouring agent ranging from 0.5 to 1.5.

19. Composition according to any one of the preceding claims, such that the goniochromatic pigments and the additional colouring agent are present in a weight ratio of goniochromatic pigments to active material of the additional colouring agent ranging from 0.9 to 1.1.

20. Composition according to one of the preceding claims, such that it comprises a hydrophilic or lipophilic cosmetic medium.

21. Composition according to one of the preceding claims, such that it comprises water or a mixture of water and of hydrophilic organic solvent.

22. Composition according to one of the preceding claims, such that it comprises a fatty phase.

23. Composition according to one of the preceding claims, such that it comprises an ingredient chosen from oils, waxes, pasty fatty substances and gums, and mixtures thereof.

24. Composition according to one of the preceding claims, such that it contains an organic solvent.

25. Composition according to one of the preceding claims, such that it comprises a cosmetic ingredient chosen from the group formed by fillers, vitamins, thickeners, trace elements, softeners, sequestering agents, fragrances, acidifying or basifying agents, and preserving agents, and mixtures thereof.

26. Composition according to one of the preceding claims, characterized in that it is in the form of a foundation, a concealer product, a face powder, an eye-shadow or a body makeup product.

27. Cosmetic skin makeup process, such that a composition according to any one of claims 1 to 26 is applied to the skin.

28. Made-up support comprising a makeup that may be obtained according to the makeup process in accordance with claim 27 and applied to the said support, the said support being chosen from pastilles or patches that adhere to the skin.
